(19) United States
(12) Patent Application Publication
(10) Pub. No.: US 2024/0379495 A1
(43) Pub. Date: Nov. 14, 2024

(54) STRUCTURE AND METHOD FOR IN-SITU MONITORING OF THERMAL INTERFACE MATERIALS

(71) Applicant: Intel Corporation, Santa Clara, CA (US)

(72) Inventors: Smit KAPILA, Bangalore (IN); Sumod CHERUKKATE, Bangalore (IN); Abhishek SRIVASTAV, Bangalore (IN); Sandesh Geejagaaru KRISHNAMURTHY, Bangalore (IN); Sankarananda BASAK, Campbell, CA (US); Ellann COHEN, Beaverton, OR (US); Jerrod PETERSON, Hillsboro, OR (US)

(21) Appl. No.: 18/314,157

(22) Filed: May 9, 2023

(51) Int. Cl. H01L 23/373; H05K 1/02

(52) U.S. Cl. CPC H01L 23/3737; H05K 1/0203

(57) ABSTRACT

The present disclosure is directed to monitoring the integrity of the thermal interface material (TIM) of a semiconductor device directly by a monitoring component of a motherboard, a system on chip (SOC), or a remote device to measure either the electrical resistivity or capacitive property of the TIM, depending on the type of TIM being used, as a means to directly assess the thermal properties (conductivity, resistance, and/or impedance) of the TIM as it ages. In an aspect, the electrical resistivity or capacitive property of the TIM may be initially measured and charted, and thereafter, the changes in the electrical resistivity or capacitive property may be sensed by the monitoring component and, based on the delta of those changes, there may be remedial actions taken to mitigate impacts to the overall system performance and/or to prevent irreparable damage to the semiconductor device/system.

### BACKGROUND

For integrated circuit design and fabrication, the need to improve performance and lower costs are constant challenges. As transistors continue to shrink in size and are used in high-power and high-density semiconductors systems, it is becoming increasingly difficult and costly to provide effective thermal management for such high-power and high-density semiconductors systems. Electrical resistance is the main reason for the generation of heat in semiconductor devices, and high-power and high-density semiconductors systems provide less surface area to dissipate the generated heat.

When a semiconductor system overheats, the devices and components with thermal ratings less than the generated temperature may be damaged. The resulting heat-induced damage in semiconductor systems is typically irreversible. To avoid thermally-induced damage, the heat generated by the semiconductor system needs to be dissipated to its surrounding environment by a thermal transfer system.

Heat sinks are commonly used, as part of a thermal transfer system for high-power and high-density semiconductor systems, to provide additional surface area for heat dissipation. The use of heat sinks in combination with other thermal management devices, such as cooling fans, can provide the necessary heat dissipation rates for most high-power and high-density semiconductor systems. The heat-generating semiconductor devices and their associated heat sinks need to be intimately connected to provide efficient heat transfer between them, and thermal interface materials (TIM) are used to avoid having any air gaps between the semiconductor devices and their heat sinks.

By various physical mechanisms, the thermal performance of a TIM may degrade or otherwise fail over time, which, in turn, may cause the thermal transfer system to become ineffective in dissipating the heat generated by the semiconductor system. To prevent impacts to overall system performance (such as reduced system speed/responsiveness, or increased actuation of system fans and a corresponding increase in noise), or possible damage to a semiconductor system, it may be advisable to proactively monitor changes in the physical properties of a TIM to detect degradation and provide notifications for significant changes in such physical properties.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the present disclosure. The dimensions of the various features or elements may be arbitrarily expanded or reduced for clarity. In the following description, various aspects of the present disclosure are described with reference to the following drawings, in which:

FIG. 1 shows an exemplary representation of an integrated heat transfer monitoring system according to an aspect of the present disclosure;

FIG. 2 shows exemplary representations of an integrated heat transfer monitoring system according to another aspect of the present disclosure;

FIG. 3 shows an exemplary representation of a semiconductor package with a thermal interface material (TIM) according to an aspect of the present disclosure; and

FIG. 4 shows an exemplary representation of a semiconductor package with a TIM according to another aspect of the present disclosure;

FIG. 5 shows an exemplary representation of a semiconductor package with a TIM according to yet another aspect of the present disclosure;

FIG. 6 shows a simplified graphical representation of the monitoring threshold for a TIM according to an aspect of the present disclosure; and

FIG. 7 shows a simplified flow diagram for an exemplary method according to an aspect of the present disclosure.

### DETAILED DESCRIPTION

The following detailed description refers to the accompanying drawings that show, by way of illustration, specific details, and aspects in which the present disclosure may be practiced. These aspects are described in sufficient detail to enable those skilled in the art to practice the present disclosure. Various aspects are provided for devices, and various aspects are provided for methods. It will be understood that the basic properties of the devices also hold for the methods and vice versa. Other aspects may be utilized and structural, and logical changes may be made without departing from the scope of the present disclosure. The various aspects are not necessarily mutually exclusive, as some aspects can be combined with one or more other aspects to form new aspects.

As a semiconductor device is used over an extended period of time, the thermal interface material (TIM), which acts as a critical channel for efficient heat transfer from the semiconductor device to its heat sink, can be susceptible to degradation (i.e., drying, shrinking, cracking, decomposition, etc.). The TIM degradation may also be caused by mechanical movements or sudden impacts. The degradation/change in the TIM properties may lead to a reduced contact area between the TIM and the semiconductor device and/or between the TIM and a heat sink.

The TIM degradation will inevitably lead to reduced heat transfer efficiency in the thermal transfer system since the TIM is a vital component of the thermal transfer system. The accumulated heat in the semiconductor device during use can lead to a deteriorating user experience (e.g., PC slowdown, lagging in games or videos, increased actuation of system fans and a corresponding increase in noise, and other performance effects). Without remedial action, in the worst-case scenario, the lack of timely TIM “refresh” can lead to CPU damage (i.e., burnout) and failures (i.e., random freezing), and the improper functioning of components. The remedial actions may include providing additional cooling (e.g., an auxiliary fan or reducing ambient temperature), part replacement, or other technical servicing.

According to the present disclosure, the “health” or integrity of the TIM may be directly monitored by a component of a motherboard, a system on chip (SOC), or remotely (i.e., an internal or external monitoring device) to measure either the electrical resistivity or capacitive property of the TIM, depending on the type of TIM being used, as a means to directly assess the heat transfer properties (thermal conductivity, resistance, and/or impedance) of the TIM. In an aspect, the electrical resistivity or capacitive property of the TIM may be initially measured and charted, and thereafter, the changes in the electrical resistivity or capacitive property may be sensed by the monitoring component. Based on the delta of those changes, there may be remedial actions taken (e.g., forcing a lower thermal design power (TDP), working at a lower voltage, alert notification to the user, etc.) to minimize system performance impacts and/or to prevent irreparable damage to the semiconductor device/system.

In a further aspect, the monitoring of the electrical resistivity or capacitive property of TIM may be performed in-situ by using circuit pathways through components and electrical connects that may be pre-existing components/connections in a semiconductor package, such as a metal heat sink or cold plate and/or power/ground connections. There is no need for external probes/devices or connections to perform the monitoring of the TIM.

The present disclosure provides a TIM monitoring solution that uses an integrated heat transfer monitoring system for a semiconductor package including at least one semiconductor device coupled to a TIM, a circuit pathway passing through the TIM and connecting to a print circuit board (PCB), and a monitoring device coupled to the circuit pathway providing resistance or capacitance measurements for the TIM.

The present disclosure is also directed to a method that includes providing a semiconductor package with a TIM that transfers heat from a semiconductor device to an external environment, forming a circuit pathway through the TIM, and performing insitu monitoring of the TIM to identify changes in its operating electrical properties.

The present disclosure is further directed to a monitoring circuit including a semiconductor package with a TIM having predetermined electrical properties, a first electrical connection between the TIM and a power source, a second electrical connection between the TIM and a ground connection on a PCB, and a circuit pathway comprising the first and second electrical connections and a monitoring device coupled the circuit pathway to monitor changes in the TIM electrical properties during operations.

The technical advantages of the present disclosure include, but are not limited to:
(i) providing an internal/in-situ scheme to monitor the physical properties of TIM;
(ii) providing a cost-effective TIM monitoring solution to detect TIM damage/degradation that requires little or no structural modifications or additions, i.e., no impact on SOC design, fabrication, processing, etc.;
(iii) providing a TIM monitoring solution that applies to the most commonly used TIMs; and
(iv) providing a TIM monitoring solution for improved sustainability and reduced carbon footprint by extending the lifetime of semiconductor devices and systems.

To more readily understand and put into practical effect the present structures and method for in-situ monitoring of TIMs, which may be used for improving sustainability and performance, particular aspects will now be described by way of examples provided in the drawings that are not intended as limitations. The advantages and features of the aspects herein disclosed will be apparent through reference to the following descriptions relating to the accompanying drawings. Furthermore, it is to be understood that the features of the various aspects described herein are not mutually exclusive and can exist in various combinations and permutations. For the sake of brevity, duplicate descriptions of features and properties may be omitted.

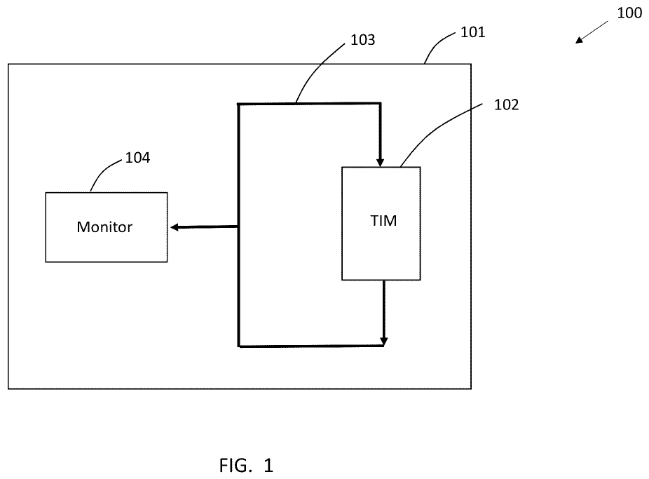

FIG. 1 shows an exemplary representation of an integrated heat transfer monitoring system 100 for an electronic component 101 according to an aspect of the present disclosure. The heat transfer monitoring system 100 may include a thermal interface material (TIM) 102 that is a part of circuit pathway 103, which is an integrated part of the electronic component 101 and is coupled to a monitoring device 104. In an aspect, the electronic component 101 may be a system-on-chip (SOC), a semiconductor package, a motherboard, or an electronic device. The electronic component 101 may be actively or passively cooled.

In another aspect, the monitoring device 104 may be incorporated in a temperature sensing component (not shown) of the electronic component 101. In an aspect, the sensing component or sensor provides monitoring of the electrical properties of a TIM. The monitoring device 104 may include a resistivity sensor and may perform calculations using Ohm's law, and/or a voltage sensor to measure capacitance or voltage difference. The electronic component 101 may have a junction temperature limit, which is the maximum allowed internal temperature of a semiconductor device (not shown) in use in the electronic component 101. During the operation of the electronic component 101, the junction temperature (Tj) is typically higher than the temperature of the electronic component's exterior and the packaging temperature.

The circuit pathway 103 passes through the TIM 102, which will have an electrical resistivity or capacitive property, that may change and be measured by the monitoring device 104 (“TIM Measured Values”) and compared with predetermined “baseline” values and predetermined thresholds for the electrical resistivity or capacitive property of the TIM 102 (“TIM Predetermined Values”). The TIM Predetermined Values for the electrical resistivity or capacitive property of the TIM 102 will be known, i.e., obtained from published results or during testing of prototypes of the electronic component 101. The TIM Predetermined Values may be correlated against the Tj and may be stored in an onboard memory component (not shown) of the monitoring device 104 or separately on the electronic component 101. The monitoring device 104 may be provided with the thermal and electrical properties of the TIM 102 and may take appropriate action (e.g., generate a TIM degradation alert or signal for the system to reduce the TDP to prevent the electronic component 101 from exceeding its Tj limit.

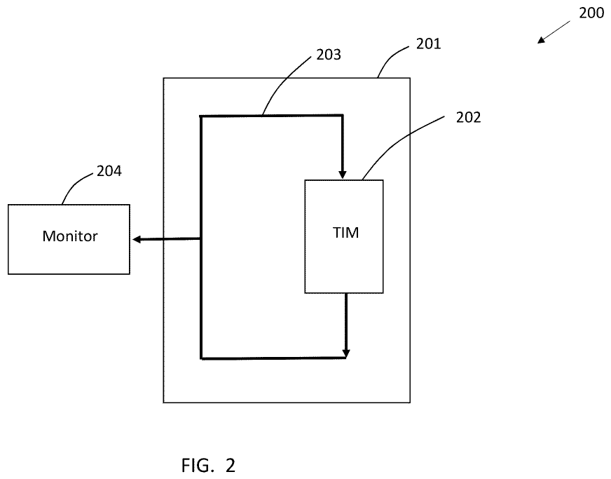

FIG. 2 shows another exemplary representation of an integrated heat transfer monitoring system 200 for an electronic component 201 according to another aspect of the present disclosure. The heat transfer monitoring system 200 may include a thermal interface material (TIM) 202 that is a part of circuit pathway 203, which is an integrated part of the electronic component 201 and is coupled to a monitoring device 204. In an aspect, the electronic component 201 may be a system-on-chip (SOC) or a semiconductor package. In another aspect, the monitor device 204 may be a sensor component on a motherboard (not shown) or possibly at a remote location. For example, the electronic component 201 may provide certain data periodically through a telemetry data collection process (i.e., automatic measurements and wireless transmissions of data to and from remote sources). The telemetry data collection process may provide monitoring throughout a product lifetime for the TIM 202 and other components of the electronic component 201, which may include performance data, sharable logs, etc.

The circuit pathway 203 passes through the TIM 202, which, similar to the aspect shown in FIG. 1, will have an electrical resistivity or capacitive property, that may have TIM Measured Values measured by the monitoring device 204 during operations of the electronic component 201 and compared with the TIM Predetermined Values for the electrical resistivity or capacitive property of the TIM 202 as a pre-defined TIM baseline resistance/capacitance model. The TIM Predetermined Values for the electrical resistivity or capacitive property of the TIM 202 may be obtained from published results or during the testing of prototypes of the electronic component 201. The TIM Measured Values may be correlated against the Tj and may be stored as a TIM model in an onboard memory component (not shown) of the monitoring device 204 or separately on the electronic component 201.

In an aspect, the monitoring device 204 may include software, built-in logic, or an application working with the operating system software to provide the TIM Measured Values.

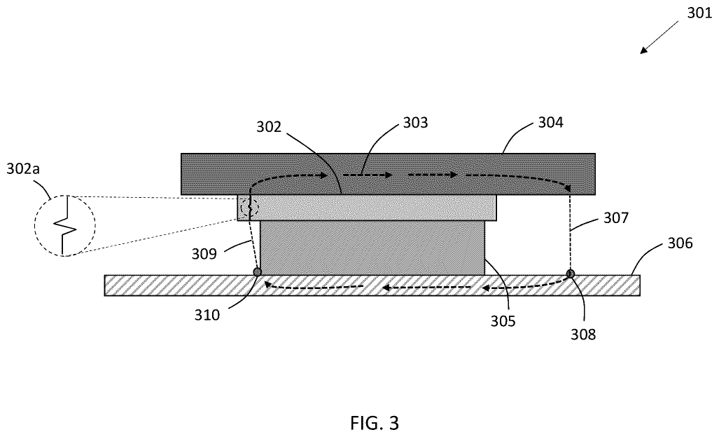

FIG. 3 shows an exemplary representation of a semiconductor package 301 with a thermal interface material (TIM) 302 according to an aspect of the present disclosure. An upper surface of the TIM 302 may be coupled to a heat dissipation/spreader component 304 (e.g., heat sink, cold plate, stiffener, package lid, etc.), and a lower surface of the TIM 302 may be coupled to a semiconductor device 305 (e.g., an SOC, CPU, GPU, etc.) that is mounted on a printed circuit board (PCB) 306.

In this aspect, a circuit pathway 303 may have a pathway from the TIM 302 through the heat dissipation component 304 to an electrical connection 307. The electrical connection 307 may be a ground connection for the heat dissipation component 304, which is electrically conductive, that may be coupled to a general-purpose input/output (GPIO) 308 or uncommitted digital signal pin on the PCB 306 that may be used as an input or output (or both) and is controllable by software. In an aspect, the electrical connection 307 may be a chassis ground. The circuit pathway 303 may continue through the PCB 306 to another GPIO 310 and via another electrical connection 309 to the TIM 302 to complete the circuit. In another aspect, a GPIO (not shown) may be configured between the semiconductor device 305 and the TIM as part of the electrical connection 309.

It should be understood that the electrical connections 307 and 309, although shown as separate wire connections for representational purposes, may be provided by various wiring configurations, such as part of a metalization or routing layer.

Further to this aspect, the TIM 303 may be positioned directly between the heat dissipation component or heatsink 304 and the semiconductor device 305, which may be lidded or a bare-die package architecture that is used primarily for high-performance systems. For example, a TIM of a lidded chip package (not shown) may be referred to as an “integrated heat spreader” or IHS of the lidded chip package. In an aspect, the TIM 303 may be a solder TIM (STIM), which is typically an indium-based alloyed with various percentages of tin, bismuth, aluminum, and/or zinc, or a liquid metal TIM (LMTIM), which are typically a gallium-based alloyed with indium and/or tin. The STIMs may be solid, thin metal “foils” or “preforms” at room temperature that are formulated to melt at a desired temperature and/or pressure, whereas the LMTIMs are fully liquid at room temperature, and have a solidification temperature typically around −10° C. to +10° C., depending upon the exact alloy.

In an aspect, the TIM Predetermined Values for the electrical resistivity property of the TIM 302 may be obtained from published results or during the testing of prototypes of the electronic component 301 that are provided as part of a TIM model stored in a memory component. As shown in FIG. 3, the material type and the resistance measurement for TIM 303 is represented by the resistor symbol shown in 302a.

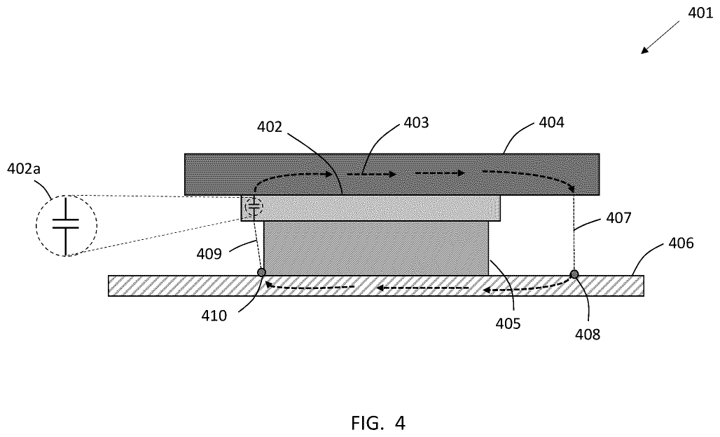

FIG. 4 shows an exemplary representation of a semiconductor package 401 with a thermal interface material (TIM) 402 according to an aspect of the present disclosure, which is similar to that shown in FIG. 3. An upper surface of the TIM 402 may be coupled to a heat dissipation/spreader component 404 (e.g., heat sink, cold plate, stiffener, package lid, etc.) and a lower surface of the TIM 402 may be coupled to a semiconductor device 405 (e.g., an SOC) that is mounted on a printed circuit board (PCB) 406.

In this aspect, a circuit pathway 403 may have a pathway from the TIM 402 through the heat dissipation component 404 to an electrical connection 407. The electrical connection 407 may be a ground connection for the heat dissipation component 404, which is electrically conductive, that may be coupled to a general-purpose input/output (GPIO) 408 or other uncommitted digital signal pin on the PCB 406 that may be used as an input or output (or both) and is controllable by software. In an aspect, the electrical connection 407 may be connected to a chassis ground. The circuit pathway 403 may continue through the PCB 406 to another GPIO 410 and via another election connection 409 to the TIM 402 to complete the circuit. It should be understood that the electrical connections 407 and 409 are shown as separate wire connections for representational purposes only, as in FIG. 3.

Further to this aspect, the TIM 402 may be positioned directly between the heat dissipation component or heatsink 404 and the semiconductor device 405, which may be lidded or have a bare-die package architecture. In an aspect, the TIM 402 may be polymer TIM (PTIM), which are typically silicone-based carriers containing thermally-conductive fillers or dielectric materials, which are typically boron nitride (BN), barium sulfide (Bas), aluminum nitride (AIN), aluminum oxide (Al2O3), hybrid polymers, and other ceramics. The thermally-conductive fillers include any one or combination of copper, silver, aluminum, and other metals, liquid metals (e.g., gallium-based alloys, etc.), and carbon materials (e.g., graphite, carbon nanotubes, carbon fiber, etc.). The PTIM may be provided in the form of grease, paste, gap fillers, and phase change materials (PCMs) that may be solid at room temp and melt at operating temperature.

In an aspect, the TIM Predetermined Values for the electrical capacitance property of the TIM 402 may be obtained from published results or during the testing of prototypes of the electronic component 401. As shown in FIG. 4, the material type and the capacitance measurement for TIM 402 is represented by the capacitor symbol shown in 402a.

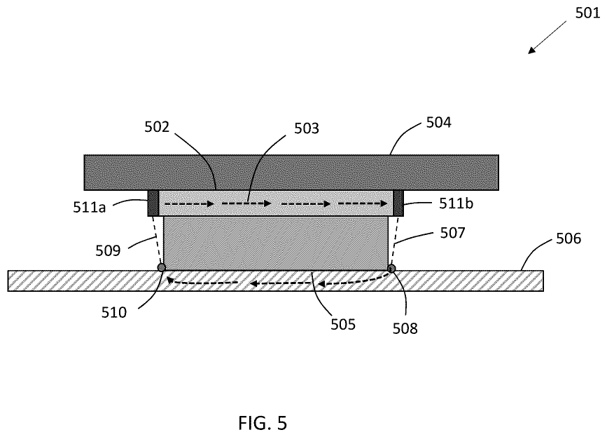

FIG. 5 shows an exemplary representation of a semiconductor package 501 with a thermal interface material (TIM) 502 according to yet another aspect of the present disclosure. An upper surface of the TIM 502 may be coupled to a heat dissipation/spreader component 504 (e.g., heat sink, cold plate, stiffener, package lid, etc.), and a lower surface of the TIM 502 may be coupled to a semiconductor device 505 (e.g., an SOC) that is mounted on a printed circuit board (PCB) 506. The TIM 502 may be a conductive STIM, which may be monitored for changes in resistance or capacitance that may occur along the length of the TIM 502.

In this aspect, a circuit pathway 503 may also provide a shorter pathway that travels through the TIM 502 from a first terminal 511a positioned at a first approximal end of the TIM 502 to a second terminal 511b positioned at a second approximal end of the TIM 502, which allows a current to flow along the TIM's length to an electrical connection 507. In addition, the electrical connection 507 may be connected to a ground connection for the semiconductor device 505, which is coupled to a general-purpose input/output (GPIO) 508 or other uncommitted digital signal pin on the PCB 506 that may be used as an input or output (or both) and is controllable by software. The circuit pathway may continue through the PCB 506 to another GPIO 510 and via another electrical connection 509 to the first terminal 511a to complete the circuit. In this aspect, the electrical connections 507 and 509 may be separate wire connections or electrical connections through the semiconductor device 505. It is within the scope of the present disclosure to place a first terminal and a second terminal at suitable first and second locations that are convenient for connections on a TIM of a semiconductor package.

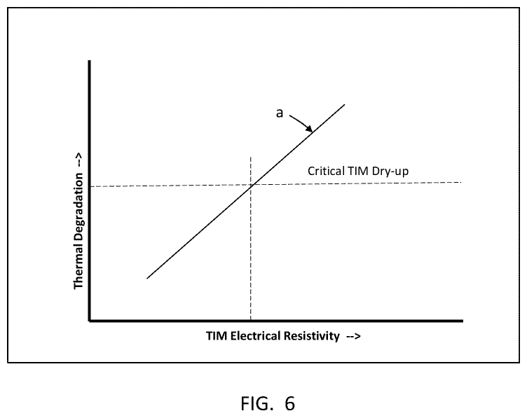

FIG. 6 shows a simplified graphical representation of the monitoring threshold for a TIM according to an aspect of the present disclosure. In this representation, the electrical resistivity of a solder TIM or STIM increases over time, and the occurrence of operating stress conditions, such as moisture, excess temperature or temperature cycling, power cycling, mechanical vibration, shock, etc. The “aging” of the STIM leads to thermal performance degradation due to various physical mechanisms (e.g., delamination from adjacent surfaces, cracking/voiding, etc.). For a typical semiconductor device, there may be a curve “a” when the TIM Measured Value reaches a critical TIM thermal degradation point or predetermined threshold for the STIM. The monitoring of the STIM may produce results as shown in Table 1 below.

TABLE 1

| | | Current Sense |
|---|---|---|
| Scenarios | Electrical Resistivity | Output/Trigger |
| Fresh STIM | <10 MΩ | Normal, No Trigger |
| Plate/STM Joint | High Z | Abnormal, Trigger |
| Damage/Mechanical | (Open Circuit) | Activated |
| Failure | | |
| Degradation Over | >50 MΩ | Abnormal, Trigger |
| Years | | Activated |

According to the present disclosure, the TIM Predetermined Values for the electrical resistivity property of a STIM may be obtained from published results or during the testing of prototypes of an electronic component, which will take into account the dimensions of the STIM. The TIM Predetermined Values may be stored in a suitable memory component and retrieved when compared with the TIM Measured Values measured by a monitoring device during operations of the electronic component.

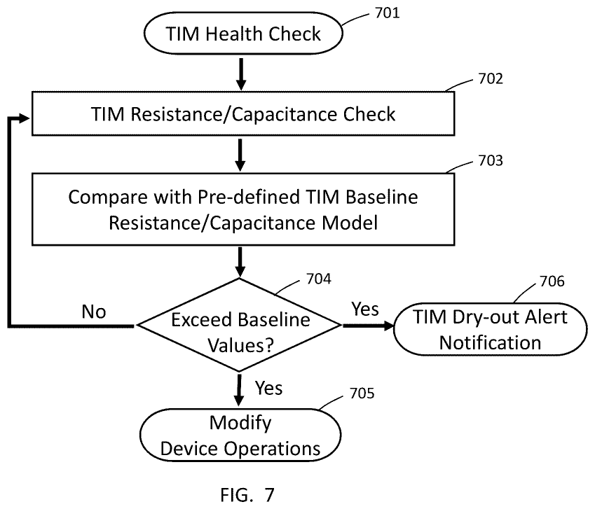

FIG. 7 shows a simplified flow diagram for an exemplary method according to an aspect of the present disclosure.

The operation 701 may be directed to initiating a TIM “health check”, which may be conducted on a periodic basis or in response to an event, such as a temperature spike.

The operation 702 may be directed to either a TIM resistance or capacitance check, based on the type of TIM used in the electronic component.

The operation 703 may be directed to accessing a pre-defined TIM baseline resistance/capacitance model that includes values that have been stored for access by a monitoring device for the electronic component and compared with a measured value.

The operation 704 may be directed to determining if the comparison shows that baseline values have been exceeded.

The operation 705 may be directed to modifying the operations of a semiconductor device or the electronic component.

The operation 706 may be directed to providing a TIM dry-out alert notification to an operator and/or the operating system program.

It will be understood that any property described herein for a particular structure and method for monitoring thermal interface material (TIM) may also hold for any electronic component using the TIMs described herein. It will also be understood that any property described herein for a specific method may hold for any of the methods described herein. Furthermore, it will be understood that for any TIM monitoring circuit and the methods described herein, not necessarily all the components or operations described will be shown in the accompanying drawings or method, but only some (not all) components or operations may be disclosed.

To more readily understand and put into practical effect the present semiconductor carrier platforms and thermal stability layers, they will now be described by way of examples. For the sake of brevity, duplicate descriptions of features and properties may be omitted.

### EXAMPLES

Example 1 provides an integrated heat transfer monitoring system including an electronic component including at least one semiconductor device coupled to a thermal interface material (TIM), a circuit pathway passing through the TIM and connecting to a printed circuit board (PCB), and a monitoring device coupled to the circuit pathway providing resistance or capacitance measurements for the TIM.

Example 2 may include the integrated heat transfer monitoring system of example 1 and/or any other example disclosed herein, for which the circuit pathway passes through a heat dissipation component.

Example 3 may include the integrated heat transfer monitoring system of example 1 and/or any other example disclosed herein, for which the circuit pathway passes through a ground connection to the PCB.

Example 4 may include the integrated heat transfer monitoring system of example 1 and/or any other example disclosed herein, for which the circuit pathway further comprises a first terminal coupled to the TIM at a first location and a second terminal coupled to the TIM at a second location.

Example 5 may include the integrated heat transfer monitoring system of example 4 and/or any other example disclosed herein, for which the circuit pathway passes from the first terminal through the TIM to the second terminal.

Example 6 may include the integrated heat transfer monitoring system of example 1 and/or any other example disclosed herein, for which the TIM is an electrically conductive material.

Example 7 may include the integrated heat transfer monitoring system of example 6 and/or any other example disclosed herein, for which the TIM is a liquid metal or a solder.

Example 8 may include the integrated heat transfer monitoring system of example 1 and/or any other example disclosed herein, for which the TIM is a dielectric material.

Example 9 may include the integrated heat transfer monitoring system of example 8 and/or any other example disclosed herein, for which the TIM is a polymer, hybrid polymer, or liquid metal.

Example 10 may include the integrated heat transfer monitoring system of example 1 and/or any other example disclosed herein, for which the monitoring device comprises a sensor for monitoring the electrical properties of the TIM and a thermal transfer rate.

Example 11 may include the integrated heat transfer monitoring system of example 1 and/or any other example disclosed herein, for which the monitoring device is provided with the thermal and electrical properties of the TIM.

Example 12 provides a method having a TIM that transfers heat from a semiconductor device, forming a circuit pathway through the TIM, and performing in-situ monitoring of the TIM to identify changes in its operating electrical properties.

Example 13 may include the method of example 12 and/or any other example disclosed herein, for which forming the circuit pathway further comprises coupling the TIM to a PCB via a ground connection.

Example 14 may include the method of example 12 and/or any other example disclosed herein, for which the TIM is formed with predetermined dimensions in a semiconductor package.

Example 15 may include the method of example 14 and/or any other example disclosed herein, for which the TIM has premeasured electrical properties that are associated with its dimensions.

Example 16 may include the method of example 15 and/or any other example disclosed herein, which further includes a step of comparing the premeasured electrical properties with the operating electrical properties of the TIM.

Example 17 may include the method of example 16 and/or any other example disclosed herein, which further includes providing a notification when comparing the premeasured electrical properties with the operating electrical properties provides a measurement that exceeds a predetermined threshold.

Example 18 may include the method of example 17 and/or any other example disclosed herein, which further includes providing instructions to the semiconductor device to operate at reduced working parameters to generate less heat when the notification is generated.

Example 19 provides a monitoring circuit that includes a semiconductor package with a TIM, for which the TIM has predetermined electrical properties, a first electrical connection between the TIM and a power source, a second electrical connection between the TIM and a ground connection on a PCB, a circuit pathway including a path that traverses the first electrical connection, the TIM and second electrical connections, and a monitoring device coupled the circuit pathway to monitor changes in the TIM electrical properties during operations.

Example 20 may include the monitoring circuit of example 19 and/or any other example disclosed herein, for which the circuit pathway further includes the second electrical connection traversing a heat dissipation component.

The term “comprising” shall be understood to have a broad meaning similar to the term “including” and will be understood to imply the inclusion of a stated integer or operation or group of integers or operations but not the exclusion of any other integer or operation or group of integers or operations. This definition also applies to variations on the term “comprising” such as “comprise” and “comprises”.

The term “coupled” (or “connected”) herein may be understood as electrically coupled or as mechanically coupled, e.g., attached or fixed or attached, or just in contact without any fixation, and it will be understood that both direct coupling or indirect coupling (in other words: coupling without direct contact) may be provided.

The terms “and” and “or” herein may be understood to mean “and/or” as including either or both of two stated possibilities.

While the present disclosure has been particularly shown and described with reference to specific aspects, it should be understood by those skilled in the art that various changes in form and detail may be made therein without departing from the spirit and scope of the present disclosure as defined by the appended claims. The scope of the present disclosure is thus indicated by the appended claims and all changes which come within the meaning and range of equivalency of the claims are therefore intended to be embraced.

Claims

What is claimed is:

1. An integrated heat transfer monitoring system comprising:
an electronic component comprising at least one semiconductor device coupled to a thermal interface material (TIM);
a circuit pathway passing through the TIM and connecting to a printed circuit board (PCB); and
a monitoring device coupled to the circuit pathway providing resistance or capacitance measurements for the TIM.

2. The integrated heat transfer monitoring system of claim 1, wherein the circuit pathway passes through a heat dissipation component.

3. The integrated heat transfer monitoring system of claim 1, wherein the circuit pathway passes through a ground connection to the PCB.

4. The integrated heat transfer monitoring system of claim 1, wherein the circuit pathway further comprises a first terminal coupled to the TIM at a first location and a second terminal coupled to the TIM at a second location.

5. The integrated heat transfer monitoring system of claim 4, wherein the circuit pathway passes from the first terminal through the TIM to the second terminal.

6. The integrated heat transfer monitoring system of claim 1, wherein the TIM is an electrically conductive material.

7. The integrated heat transfer monitoring system of claim 6, wherein the TIM is a liquid metal or a solder.

8. The integrated heat transfer monitoring system of claim 1, wherein the TIM is a dielectric material.

9. The integrated heat transfer monitoring system of claim 8, wherein the TIM is a polymer, hybrid polymer, or liquid metal.

10. The integrated heat transfer monitoring system of claim 1, wherein the monitoring device comprises a sensor for monitoring electrical properties of the TIM and a thermal transfer rate.

11. The integrated heat transfer monitoring system of claim 1, wherein the monitoring device is provided with the thermal and electrical properties of the TIM.

12. A method comprising:
providing a TIM that transfers heat from a semiconductor device;
forming a circuit pathway through the TIM; and
performing in-situ monitoring of the TIM to identify changes in its operating electrical properties.

13. The method of claim 12, wherein forming the circuit pathway further comprises coupling the TIM to a PCB via a ground connection.

14. The method of claim 12, wherein the TIM is formed with predetermined dimensions in a semiconductor package. 15 The method of claim 14, wherein the TIM has premeasured electrical properties that are associated with its dimensions.

16. The method of claim 15, further comprises a step of comparing the premeasured electrical properties with the operating electrical properties of the TIM.

17. The method of claim 16, further comprises providing a notification when the comparing the premeasured electrical properties with the operating electrical properties provides a measurement that exceeds a predetermined threshold.

18. The method claim 17, further comprises providing instructions to the semiconductor device to operate at reduced working parameters to generate less heat when the notification is generated.

19. A monitoring circuit comprising:
a semiconductor package with a TIM, wherein the TIM has predetermined electrical properties;
a first electrical connection between the TIM and a power source;
a second electrical connection between the TIM and a ground connection on a PCB;
a circuit pathway comprising a path that traverses the first electrical connection, the TIM, and second electrical connections; and
a monitoring device coupled to the circuit pathway to monitor changes in the TIM electrical properties during operations.

20. The monitoring circuit of claim 19, wherein the circuit pathway further comprises the second electrical connection traversing a heat dissipation component.
